(19) United States
(12) Patent Application Publication
(10) Pub. No.: US 2006/0074104 A1
(43) Pub. Date: Apr. 6, 2006

(54) Use of clioquinol for the therapy of alzheimer's disease

(76) Inventors: Ashley I. Bush, Sommerville, MA (US); Rudolph E. Tanzi, Hull, MA (US); Mikhal Xilinas, Athens (GR); Robert Cherny, Melbourne (AU)

Correspondence Address: STERNE, KESSLER, GOLDSTEIN & FOX PLLC, 1100 NEW YORK AVENUE, N.W., WASHINGTON, DC, 20005, US

(21) Appl. No.: 11/099,693

(22) Filed: Apr. 6, 2005

Related U.S. Application Data
(63) Continuation of application No. 09/972,913, filed on Oct. 10, 2001, now abandoned.
Application No. 09/972,913 is a continuation of application No. 09/560,887, filed on Apr. 28, 2000, now abandoned.
Application No. 09/560,887 is a continuation of application No. 09/224,953, filed on Jan. 4, 1999, now abandoned.
Application No. 09/224,953 is a continuation of application No. 09/032,777, filed on Mar. 6, 1998, now abandoned.

(51) Int. Cl. A61K 31/47

(52) U.S. Cl. 514/311

(57) ABSTRACT

The invention relates to the identification of clioquinol as a pharmaceutically therapeutic agent for treatment of Alzheimer's disease and related pathological conditions.

### BACKGROUND OF THE INVENTION

1. Field of the Invention

This invention is in the field of medicinal chemistry. In particular, the invention is related to the use of clioquinol for therapy of Alzheimer's disease.

2. Related Art

Polymers of Abeta (β), the 4.3 kD, 39-43 amino acid peptide product of the transmembrane protein, amyloid protein precursor (APP), are the main components extracted from the neuritic and vascular amyloid of Alzheimer's disease (AD) brains. Aβ deposits are usually most concentrated in regions of high neuronal cell death, and may be present in various morphologies, including amorphous deposits, neurophil plaque amyloid, and amyloid congophilic angiopathy (Masters, C. L., et al, EMBO J. 4:2757 (1985); Masters, C. L. et al., Proc. Natl. Acad Sci. USA 82: 4245 (1985)). Growing evidence suggests that amyloid deposits are intimately associated with the neuronal demise that leads to dementia in the disorder.

The presence of an enrichment of the 42 residue species of Aβ in these deposits suggests that this species is more pathogenic. The 42 residue form of Aβ (Aβ₁₋₄₂), while a minor component of biological fluids, is highly enriched in amyloid, and genetic studies strongly implicate this protein in the etiopathogenesis of AD. To date, the cause of Aβ deposits is unknown, although it is believed that preventing these deposits may be a means of treating the disorder.

Studies into the neurochemical vulnerability of Aβ to form amyloid have suggested altered zinc and [H⁺] homeostasis as the most likely explanations for amyloid deposition. Aβ is rapidly precipitated under mildly acidic conditions in vitro (pH 3.5-6.5) (Barrow, C. J. & Zagorski, M. G., Science 253:179-182 (1991); Fraser, P. E., et al., Biophys. J. 60:1190-1201 (1991); Barrow, C. J., et al., J. Mol. Biol. 225:1075-1093 (1992); Burdick, D., J. Biol. Chem. 267:546-554 (1992); Zagorski, M. G. & Barrow, C. J., Biochemistry 31:5621-5631 (1992); Kirshenbaum, K. & Daggett, V., Biochemistry 34:7629-7639 (1995); Wood, S. J., et al., J. Mol. Biol. 256:870-877 (1996)). Recently, it has been shown that the presence of certain biometals, in particular redox inactive Zn²⁺ and, to a lesser extent, redox active Cu²⁺ and Fe³⁺, markedly increases the precipitation of soluble Aβ (Bush, A. T., et al., J. Biol. Chem. 268:16109 (1993); Bush, A. I., et al., J. Biol. Chem. 269:12152 (1994); Bush, A. I., et al., Science 265:1464 (1994); Bush, A. I., et al., Science 268:1921 (1995)). At physiological pH, Aβ₁₋₄₀ specifically and saturably binds Zn²⁺, manifesting high-affinity binding (K_{D}=107 nM) with a 1:1 (Zn²⁺:Aβ) stoichiometry, and low affinity binding (K_{D}=5.2 μM) with a 2:1 stoichiometry.

Clioquinol (iodochlorhydroxyquin, 5-chloro-7-iodo-8-hydroxyquinoline, MW 305.5) is a USP drug that chelates zinc [K(Zn)=12.5, K(Cu)=15.8, K(Ca)=8.1, K(Mg)=8.6], is hydrophobic, has a low general toxicity profile, and crosses the blood brain barrier (Padmanabhan et al., 1989). It has been used as an oral anti-amebic antibiotic, and as a topical antibiotic.

It has been demonstrated that clioquinol is rapidly absorbed from the gut of rats and mice where blood levels reached≈1-10 μM within one hour of ingestion (Kotaki et al., J Pharmacobiodyn, 6(11):881-887 (1983)). Since the drug is hydrophobic, it passes rapidly into the brain, and then is rapidly excreted, so that a bolus dose of clioquinol is almost completely removed from the brain within three hours. It appears to be safe in many mammalian species, including rat and mouse (Tateishi et al., 1972; Tateishi. et al., 1973), and is still used as a veterinary antibiotic (Entero Vioform).

Clioquinol was withdrawn from use as an oral antibiotic for humans in the early 1970's when its ingestion in Japan was linked to a mysterious condition called subacute myelo-optic neuritis (SMON), a condition that resembles subacute combined degeneration of the cord caused by vitamin B12 deficiency. The mechanism of SMON has never been elucidated, but in the 1970's a considerable literature developed exploring the pathophysiology of clioquinol ingestion (Tateishi et al., 1972; Tateishi et al., 1973). Several reports have demonstrated that clioquinol complexes with zinc in the brain, especially in areas enriched in synaptic vesicular zinc such as the temporal lobe (Shiraki, H. Handbook of Clinical Neurology, Vol. 37 (1979)). Indeed, over ingestion of clioquinol has been reported to induce amnesia in humans (Shiraki, H. Handbook of Clinical Neurology, Vol: 37 (1979)).

### SUMMARY OF THE INVENTION

The first aspect of the invention relates to a method for the therapy of amyloidosis comprising administering to a patient in need thereof an effective amount of clioquinol. Clioquinol may be administered alone or in combination with Vitamin B12 and/or trace metals.

The amount of clioquinol administered may be between about 10-250 milligram per kilogram body weight of the patient Preferably, however, 3-15 mg/kg body weight, and most preferably 5-10 mg/kg bodyweight is administered.

Vitamin B12 may be administered at any amounts customary for Vitamin B12 supplementation. However, it is preferred to administer about 5-15 milligram, most preferably 7-10 milligram, Vitamin B12 per kilogram body weight of said patient per day if administered orally. When administered intramuscularly, about 50-150 microgram, most preferably 70-100 microgram, Vitamin B12 per kilogram body weight of said patient per month is sufficient.

Trace metals may be supplemented at the customary supplementation levels up to the limits of toxicity. Trace metal administration as well as Vitamin B12 supplementation may be done concurrently with the administration of clioquinol or subsequent thereto during a wash out period.

Clioquinol may be administered alone or in combination with Vitamin B12 and/or the trace metals, parenteraly, e.g. intradermaly, or orally. It is preferred that clioquinol administration be carried out intermittently, not allowing sustained levels of the drug concentration for extended periods of time.

The duration of therapy may last up to 10 years, preferably 12 months in case of moderately affected individuals. In case of severely affected patients with low quality of life, 1-21 days, preferably 14 days, using high doses of clioquinol. The method of claim 1, wherein the therapy is carried out up to 10 years.

### BRIEF DESCRIPTION OF THE FIGURES

FIG. 1 is a graphical representation of resolubilization of Zn, Cu, or pH induced aggregates in vitro. Values are expressed as a percentage of Aβ signal after washing with TBS alone.

FIG. 2 shows extraction of Aβ from brain tissue with clioquinol. Undiluted (100%) clioquinol is 1.6 μM. S1 and S2 represent two sequential extractions from AD-affected tissue.

FIGS. 3A and 3B: FIG. 3A shows a western blot of Aβ extracted from brain tissue by various concentrations of clioquinol. FIG. 3B is a graphic representation of solubilization of Aβ by clioquinol.

FIG. 4 shows a bar graph demonstrating that clioquinol effectively dissolving Aβ aggregates. Aβ₁₋₄₀ was incubated with no metal, Zn (II), Zn (II)+clioquinol, DMSO or clioquinol (120 μM) in 20 mM HEPES, 150 mM NaCl, pH 7.4. Samples were incubated for 30 minutes at 37° C. and then centrifuged at 10,000 g for 20 minutes and the protein content of the supernatant determined using the BCA assay. Clioquinol was dissolved in DMSO prior to adding 20 μM to the samples. Clioquinol attenuated Zn-induced Aβ₁₋₄₀ aggregation. DMSO had no effect on Aβ aggregation. Results are mean±SD, n=3.

### DETAILED DESCRIPTION OF THE PREFERRED EMBODIMENTS

### DEFINITIONS

In the description that follows, a number of terms are utilized extensively. In order to provide a clear and consistent understanding of the specification and claims, including the scope to be given such terms, the following definitions are provided.

Aβ peptide is also known in the art as Aβ, β protein, β-A4 and A4. In the present invention, the Aβ peptide may be comprised of peptides Aβ₁₋₃₉, Aβ₁₋₄₀, Aβ₁₋₄₁, Aβ₁₋₄₂, and Aβ₁₋₄₃. The most preferred embodiment of the invention makes use of Aβ₁₋₄₀. However, any of the Aβ peptides may be employed according to the present invention. The sequence of Aβ peptide is found in Hilbich, C., et al., J. Mol. Biol. 228:460-473 (1992).

Amyloid as is commonly known in the art, and as is intended in the present specification, is a form of aggregated protein.

Amyloidosis is any disease characterized by the extracellular accumulation of amyloid in various organs and tissues of the body.

Aβ Amyloid is an aggregated Aβ peptide. It is found in the brains of patients afflicted with AD and DS and may accumulate following head injuries.

Zinc, unless otherwise indicated, means salts of zinc, i.e., Zn²⁺ in any form, soluble or insoluble.

Wash Out Period, unless otherwise indicated, means the relatively prolonged period between two administrations of clioquinol, during which clioquinol is cleared from patient's body. Wash out period may last between one to four weeks.

Considerable evidence now indicates that the accumulation of Aβ in the brain cortex is very closely related to the cause of Alzheimer's disease. Aβ is a normal component of biological fluids whose function is unknown, Aβ accumulates in a number of morphologies varying from highly insoluble amyloid to deposits that can be extracted from post-mortem tissue in aqueous buffer. The factors behind the accumulation are unknown, but the solubility of synthetic Aβ peptide has been systematically appraised in order to get some clues as to what kind of pathological environment could induce the peptide to precipitate.

Direct evidence has been obtained that show zinc and copper to be integral components of the Aβ deposits in the brain in AD. It is disclosed herein that clioquinol, a zinc- and copper-specific chelator, dramatically re-dissolves a significant proportion (up to 70%) of Aβ extracted from post-mortem AD affected brain tissue, compared to the amount extracted from the tissue by buffer in the absence of chelators. These data support a strategy of re-dissolving Aβ deposits in vivo by chelation with clioquinol.

The growing evidence in the art indicates that in AD patients, physiological levels of zinc aggregate Aβ and result in precipitation of the same and formation of amyloid deposits. Although one may speculate as to using chelators of zinc to prevent zinc from aggregating and precipitating Aβ in the brain, it is not clear whether Aβ amyloids can be dis-aggregated and redissolved into the biological fluid in the surrounding brain milieu. Therefore, the present discovery that clioquinol is capable of dissolving Aβ amyloid is a significant step towards designing a drug for the therapy of Alzheimer's Disease, and perhaps Dawn Syndrom and other conditions caused by formation of such aggregates, causing amyloidosis.

Accordingly, the present invention is directed to clioquinol as such therapeutic agent. Results of experiments, presented herein, demonstrate that clioquinol is capable of dis-aggregating Aβ amyloid deposits.

Clioquinol (iodochlorhydroxyquin, 5-chloro-7-iodo-8-hydroxyquinoline, MW 305.5) is a USP drug that chelates zinc [K(Zn)=12.5, K(Cu)=15.8, K(Ca)=8.1, K(Mg)=8.6], is hydrophobic, has a low general toxicity profile, and crosses the blood brain barrier (Padmanabhan et al., 1989). It therefore possesses some of the ideal prototypic properties for an agent for solubilization of zinc-assembled Aβ deposits in vivo. It has been used as an oral anti-ameobic antibiotic, and as a topical antibiotic.

It has been demonstrated that clioquinol is rapidly absorbed from the gut of rats and mice where blood levels reached≈1-10 μM within one hour of ingestion (Kotaki et al., J Pharmacobiodyn, 6(11):881-887 (1983)). Since the drug is hydrophobio, it passes rapidly into the bran and then is rapidly excreted, so that a bolus dose of clioquinol is almost completely removed from the brain within three hours. It appears to be safe in many mammalian species; including rat and mouse (Tateishi et al., 1972; Tateishi et al., 1973), and is still used as a veterinary antibiotic (Entero Vioform).

Clioquinol was withdrawn from use-as an oral antibiotic for humans in the early 1970's when its ingestion in Japan was lined to a mysterious condition called subacute myelo-optic neuritis (SMON), a condition that resembles subacute combined degeneration of the cord caused by vitamin. B12 deficiency. The mechanism of SMON has never been elucidated, but in the 1970's a considerable literature developed exploring the pathophysiology of clioquinol ingestion (Tateishi et al., 1972; Tateishi et al., 1973). Several reports have demonstrated that clioquinol complexes with zinc in the brain, especially in areas enriched in synaptic vesicular zinc such as the temporal lobe (Shiraki H. Handbook of Clinical Neurology, Vol. 37 (1979)). Indeed, over ingestion of clioquinol has been reported to induce amnesia in humans (Shiraki, H. Handbook of Clinical Neurology, Vol. 37 (1979)).

Clioquinol has a relatively safe profile in mice, and there is a large literature on its pharmacology in this animal. It is disclosed herein data regarding its ability to specifically chelate zinc from Aβ deposits in vitro (induced aggregates and brain samples). Based on the in vitro data described herein it is reasonably expected that the low concentrations of clioquinol shown to be effective in resolubilizing Aβ in the present invention may avoid the adverse SMON effect noted above. Thus, given its other pharmacological properties, clioquinol holds promise as an effective agent in the treatment of AD in humans.

It has been found that there is a clioquinol concentration “window” within which the Aβ aggregates are dissolved. Increasing the concentration of clioquinol above the window not only is toxic to the patient but also sharply drops the dissolution effect of clioquinol on the Aβ amyloid. Similarly, amounts below that of the window are too small to result in any dissolution.

Therefore, for each given patient, the attending physician need be mindful of the window effect and attend to varying the dosages of clioquinol so that during the course of administration, clioquinol concentrations would be varied frequently to randomly allow achieving the most effective concentration for dissolving Aβ amyloid deposits in the given patient

It is, therefore, desired that the plasma levels of clioquinol not be steady state, but be kept fluctuating between 0.01 μM but not greater than 2 μM. Since the drug is absorbed to reach peak plasma levels within 30 minutes of oral ingestion, and since the excretion half life is about 1-3 hours, the best way to dose the patient is with oral doses no more often than every three hours, preferably every six hours or eight hours, but as infrequently as once every day or once every two days are expected to be therapeutic.

An oral dose of 200 mg/kg achieves 5 μM plasma levels in rats, and 10-30 μM in dogs. An oral dose of 500 mg/kg achieves 20-70 μM in monkeys. The drug is freely permeable into the brain and is rapidly excreted.

Therefore, in humans, it is expected that a plasma level of 0.5 μM would be achieved within 30 minutes of ingesting 10 mg/kg body weight. In a 70 kg person this is 700 mg of clioquinol. Therefore, a dose of 700 mg four times a day (2800 mg/day) would be therapeutic.

However, sustained treatment with doses of clioquinol at a dose as low as 10 mg/kg/day causes the neurological side effect, subacute myelo-optic neuritis. Therefore, dosage that high is undesirable. This is equivalent to 700 mg/day. The side effect is believed to be due to loss of vitamin B12. Therefore, co-therapy with vitamin B12 100 μM/day orally or, preferably, 1000 μM/month intramuscularly, is to be administered with clioquinol treatment to abolish this side effect.

To minimize the chances of this side effect, a lower dose of clioquinol can also be used—100 mg, three or four times a day would achieve peak plasma levels of about 0.1 μM, and is likely to be therapeutic without putting the patient at risk for neurological side effects. Nevertheless, co-administration of Vitamin B12 should be mandatory.

For the treatment of moderately affected or severely affected patients, where risking the neurological side effects is less of a concern since the quality of their life is very poor, the patient may be put on a program of treatment (after informed consent) consisting of high dose clioquinol for 1 to 21 days, but preferably no more than 14 days, followed by a period of low dose therapy for seven days to three months. A convenient schedule would be two weeks of high dose therapy followed by two weeks of low dose therapy, oscillating between high and low dose periods for up to 12 months. If after 12 months the patient has made no clinical gains on high/low clioquinol therapy, the treatment should be discontinued. All regiments would be accompanied by Vitamin B12 co-therapy.

Another typical case would be the treatment of a mildly affected individual. Such a patient would be treated with low dose clioquinol for up to 12 months. If after 6 months no clinical gains have been made, the patient could then be placed on the high/low alternation regimen for up to another 12 months.

Accordingly, the present invention contemplates compositions such as pharmaceutical compositions comprising an active agent and one or more pharmaceutically, acceptable carriers and/or diluents. The active agent may be clioquinol or a combination of clioquinol and another metal chelating compound.

The pharmaceutical forms containing the active agents may be administered in any convenient manner such as by intravenous, intraperitoneal, subcutaneous, rectal, implant, transdermal, slow release, intrabuccal, intracerebral or intranasal administration. Generally, the active agents need to pass the blood brain barrier and may have to be chemically modified to facilitate this or be administered directly to the brain or via other suitable routes. For injectable use, sterile aqueous solutions (where water soluble) are generally used or alternatively sterile powders for the extemporaneous preparation of sterile injectable solutions may be used. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The preventions of the action of microorganisms can be brought about by various antibacterial and antifungal agents, for example, parabens, chlorobutanol phenol sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active agents in the required amount in the appropriate solvent with various of the other ingredients enumerated above, as required, followed by sterilization by, for example, filtration or irradiation In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof. Preferred compositions or preparations according to the present invention are prepared so that an injectable dosage unit contains enough clioquinol to raise the plasma concentration of clioquinol in the subject, the patient, to about between 0.01-1 μM.

When the active agents are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 10 mg and 1000 mg, preferably 50-500 mg, and most preferably 200-500 mg of clioquinol.

The tablets, troches, pills, capsules and the like may also contain other components such as listed hereafter. A binder such as gum, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed: In addition, the active compound(s) may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail

The principal active ingredient is compounded for convenient and effective administration in effective amounts with a suitable pharmaceutically acceptable carrier in dosage unit form as hereinbefore disclosed. A unit dosage form can, for example, contain the principal active compound in amounts ranging from 10 mg to about 2000 mg. Alternatively, amounts ranging from 1 mg/kg body weight to above 20 mg/kg body weight may be administered. Preferably, however, the amount of the principal active ingredient, clioquinol, is about 3-15 mg/kg body weight, most preferably about 5-10 mg/kg body weight. The amounts may be for individual active agents or for the combined total of active agents.

Compositions of the present invention include all compositions wherein the compounds of the present invention are contained in an amount which is effective to achieve their intended purpose. They may be administered by any means that achieve their intended purpose. The dosage administered will depend on the age, health, and weight of the recipient, kind of concurrent treatment, if any, frequency of the treatment, and the nature of the effect desired. The dosage of the various compositions can be modified by comparing the relative in vivo potencies of the drugs and the bioavailability using no more than routine experimentation.

The pharmaceutical compositions of the invention may be administered to any animal which may experience the beneficial effects of the compounds of the invention. Foremost among such animals are mammals, e.g., humans, although the invention is not intended to be so limited.

The following examples are provided by way of illustration to further describe certain preferred embodiments of the invention, and are not intended to be limiting of the present invention, unless specified.

### EXAMPLES

Dissolving Clioquinol

In order to obtain a solution of clioquinol in PBS, the following protocol was followed: 5.3 grams of clioquinol was suspended with agitation in 200 milliliter of n-decane. The undissolved material was settled, air dried, and weighed, based on which it was determined that only 2% of the clioquinol had dissolved in the n-decane. 100 milliliter of the supernatant (light yellow) was agitated in 100 milliliter of PBS, pH 7.4. Next, the phases were allowed to separate. The lower phase (PBS) was collected and filtered to remove the residue which had formed at the phase interface upon extraction with the organic solvent. The concentration of clioquinol in the PBS was determined to be 800 nanamolar. This number was arrived at based on two assumptions: (1) 2% of the clidquinol was dissolved in the n-decane; and (2) the partitioning coefficient is 1/1750 with PBS at 1:1 mixture of n-decane to clioquinol.

### Example 1

Aβ Aggregates by C Resolubilization of Metal-Induced Lioquinol

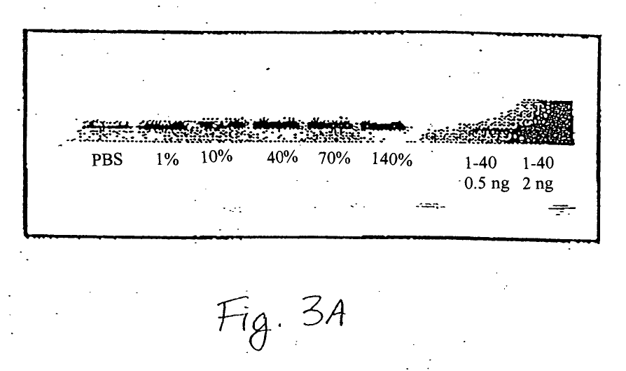

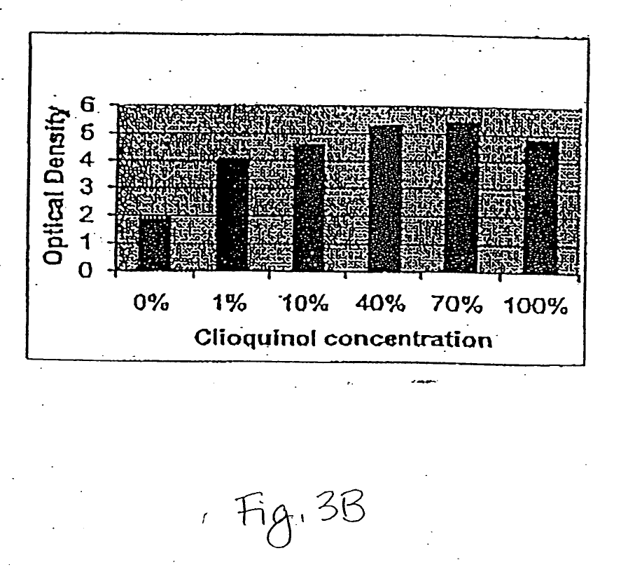

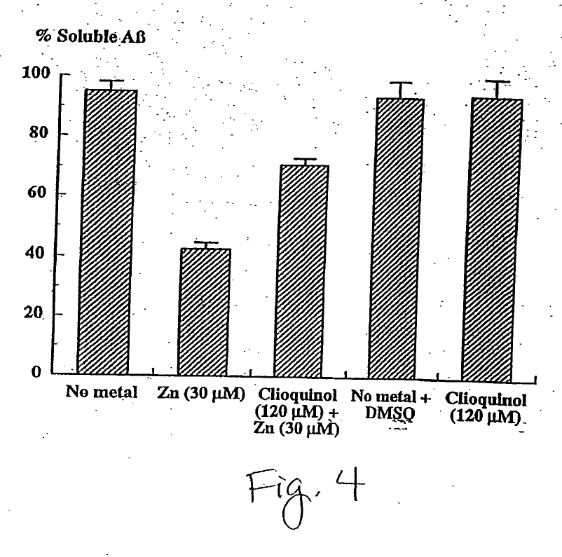

Aβ (10 ng/well in TBS) aggregation was induced by addition of ZnCl₂ (25 μM), CuCl₂ (5 μM) or acidic conditions (pH 5.5). Aggregates were transferred to a 0.2μ nylon membrane by filtration. The aggregates were then washed (200 μl/well) with TBS alone, TBS containing 2 μM EDTA, or TBS containing 2 μM clioquinol. The membrane was fixed, probed with the anti-Aβ monoclonal antibody 6E10, and developed for exposure to ECL-film. FIG. 3A shows relative signal strength as determined by transmittance analysis of the ECL-film, calibrated against known amounts of the peptide. Values are expressed as a percentage of Aβ signal after washing with TBS alone.

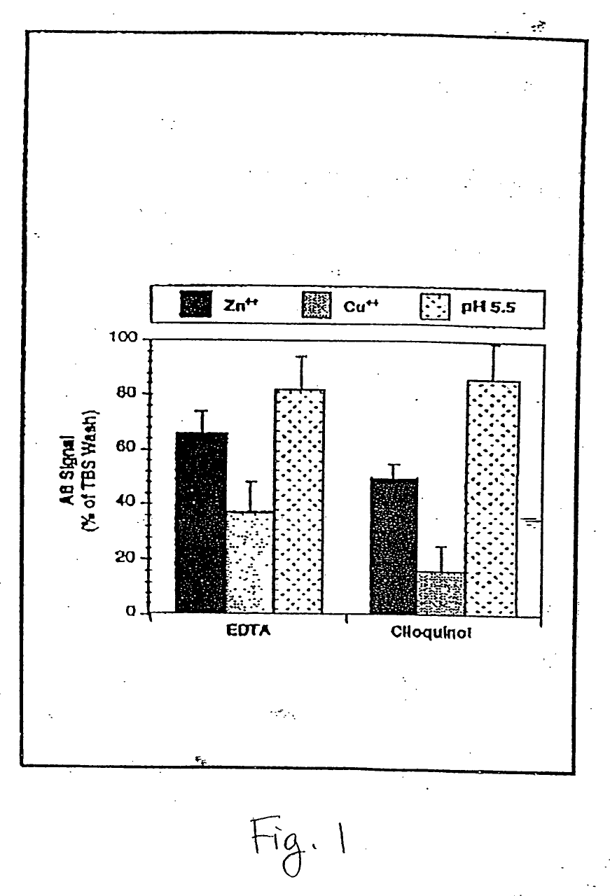

Both EDTA and clioquinol treatments were more effective than TBS alone at resolubilizing the retained (aggregated) Aβ when the peptide was precipitated by Zn or Cu (see FIG. 1). When Aβ was precipitated by pH 5.5 however, it was not resolubilized more readily by either chelator compared to TBS washing alone. The pH 5.5 precipitate contains a much greater proportion of beta-sheet amyloid than the Aβ precipitates formed by Zn or Cu.

### Example 2

Aβ Extraction from Human Brain Post-Mortem Samples

Zinc-mediated Aβ deposits in human brain have been recently characterized (Cherny, R. A., et al., Soc. Neurosci Abstr. 23:(Abstract) (1997)). Also, it was recently reported that there is a population of water-extractable Aβ deposit in the AD-affected brain (duo, Y-M., et al., J. Biol. Chem. 271:4077-81 (1996)). It was hypothesized that homogenization of brain tissue in water may dilute the metal content in the tissue, therefore, lowering the putative zinc concentration in Aβ collections, and liberating soluble Aβ subunits by freeing Aβ complexed with zinc [Zn(II)].

To test this hypothesis, the brain tissue preparation of Kuo and colleagues was replicated, but phosphate-buffered saline pH 7.4 (PBS) was substituted as the extraction buffer, achieving similar results. Highly sensitive and specific anti-Aβ monoclonal antibodies (Ida et al., (1996)) were used to assay Aβ extraction by western blot. Next, the extraction of the same material with PBS was repeated in the presence of clioquinol and determined that the presence of clioquinol increased the amount of Aβ in the soluble extract several-fold (FIGS. 1, 2, 3A, and 3B).

The amount of Aβ detected in the pellet fraction of each sample is correspondingly lower (data not shown), indicating that the effect of clioquinol is upon the disassembly of the Aβ aggregate, and not by inhibition of an Aβ-cleaving metalloprotease. The extraction of sedimentable Aβ into the soluble phase correlated only with the extraction of zinc from the pellet, and not with any other metal assayed. Examination of the total amount of protein released by the treatment revealed that chelation was not merely liberating more proteins in a non-specific manner (data not shown).

### Example 3

### Resolubilization of Aβ by Clioquinol

Resolubilization of In Vitro Metal-Induced Aβ Aggregates

First, the efficacy of clioquinol's ability to resolubilize Aβ aggregates, formed in vitro by the action of Cu(II) or Zn(II) upon Aβ1-40, was examined. FIG. 1 shows resolubilization of metal-induced Aβ aggregate by chelators. Aβ (10 ng/well in buffered saline) aggregation was induced by addition of ZnCl₂ (5 μM) or acidic conditions (pH 5.5). Aggregates were transferred to a 0.2μ nylon membrane by filtration. The aggregates were then washed (200 μl/well) with TBS alone, TBS containing 2 μM EDTA or TBS with 2 μM clioquinol. The membrane was then fixed, probed with anti-Aβ monoclonal antibody 6E10 and developed for exposure to ECL-film.

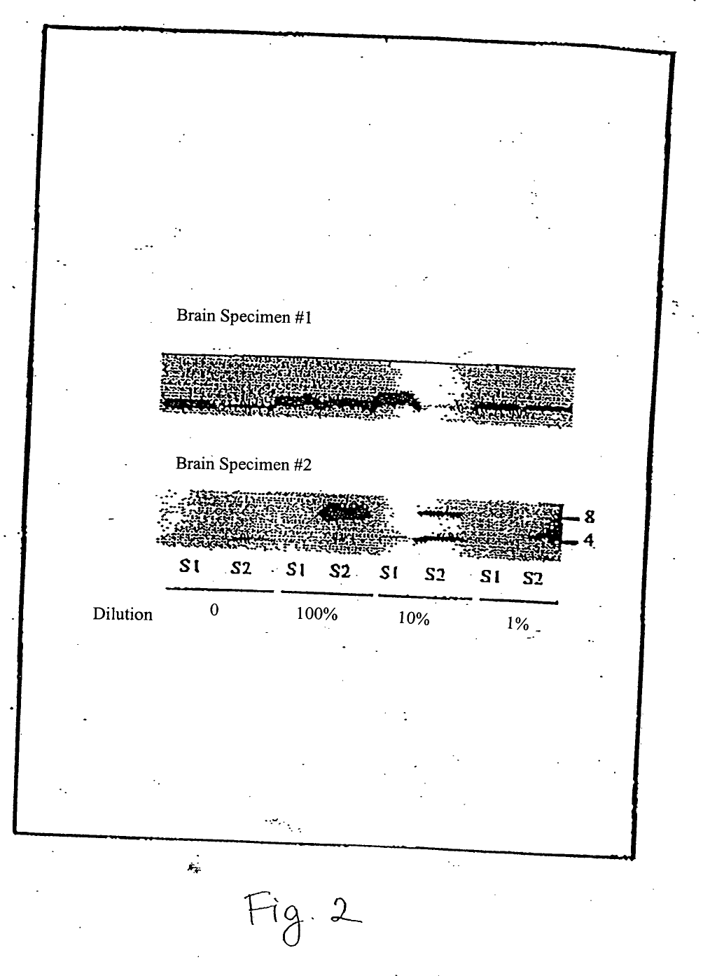

FIG. 2 shows the relative signal as determined by densitometric analysis of the ECL-film, calibrated against known amounts of the peptide. Values are expressed as a % of Aβ signal remaining on the filter after washing with TBS alone. Clioquinol is hydrophobic, so that the reagent must first be solubilized in an organic solvent, and then partitioned into the aqueous buffer according to established protocols (Padmanabhan et al., 1989).

It was found that, like EDTA (FIG. 1), clioquinol significantly resolubilized precipitated Aβ. CU(II) partially precipitates Aβ1-40 (Bush, A. I., et al., Science 268:1921 (1995)) at pH 7.4. It was determined that EDTA (2 μM) resolubilized 35% of a Zn(II)-induced Aβ precipitate, 60% of a Cu(II)-induced precipitate, and 15% of a pH 5.5-induced precipitate. In contrast, clioquinol (2 μM) was more effective at resolubilizing the Zn(II)- and Cu(II)-induced Aβ precipitates (50%, and 85%, respectively); but was also ineffective at resolubilizing the pH 5.5 precipitate (10%). Since the aggregate at pH 5.5 is predominantly β-sheet (Wood, S. J. et al., J. Mol. Bio., 256:870-877 (1996)), these data indicate that the resolubilization of Aβ by clioquinol/EDTA is likely to be due to specific chelation effects.

Extraction of Aβ from Samples of AD-Affected Brains

Next, the ability of clioquinol to extract Aβ deposits from human brain was determined. It was found that clioquinol efficiently increases the resolubilization of Aβ, compared to the amount of Aβ resolubilized from the pellet fraction of brain homogenate by PBS alone. FIG. 3 shows the effect of clioquinol upon the extraction of Aβ from AD-affected brain. Fragments of prefrontal cortex from individual post-mortem samples with the histopathological diagnosis of AD were homogenized in PBS, ph 7.4, and then pelleted after centrifugation.

The pellets were then washed with agitation twice for 30 minutes, 4° C., with PBS or PBS containing clioquinol (100%=0.8 μM clioquinol). The suspension was then pelleted (10,000 g for 30 minutes) and the supernatant removed (S1) for western blot analysis using Aβ-specific antibodies. The pellet was treated a second time in this experiment with agitation and centrifugation, and the second supernatant (S2) analyzed. The data show typical results by western blot.

In agreement with earlier findings which showed that the optimal concentration of chelator for the extraction of Aβ is idiosyncratic from case to case, and that there is a paradoxical diminution of Aβ extraction when the chelator concentration rises above the optimum, it was found that optimal clioquinol concentrations for Aβ resolubilization vary in a similar manner (e.g., Specimen #1=0.08 μM, #2=0.8 μM). It was also observed that apparently dimeric Aβ was more frequently observed on SDS-PAGE, and that in these cases (e.g., Specimen #2) the first wash did not resolubilize much Aβ, but the second wash was very efficient at resolubilizing the peptide. It is surmised that the pellet mass may be coated with adventitial, non-Aβ, proteins that are removed by the first wash, allowing the second treatment access to the Aβ collection. Indeed, further studies have shown that both sustained (for 16 hours) and repeated exposure to the chelator increases the resolubilization of Aβ significantly.

FIGS. 3A and 3B show the western blot and accompanying densitometric analysis of resolubilization of Aβ from AD-affected brain. FIG. 3A is a western blot showing the effect of clioquinol upon the resolubilization of Aβ from AD-affected brain. In this study, the brain specimen (from a different case than that of FIG. 2) was homogenized by the modified method of Kuo and colleagues, as described in Example 2, above. In this case a dose-dependent response to clioquinol was observed. Synthetic peptide standards that were used to calibrate densitometric quantification are shown in the two right-most lanes.

FIG. 3B is a chart showing densitometry performed upon the results in FIG. 3A, above. Proportional change in the amount of Aβ recovered in the extraction of Aβ by clioquinol from human brain is shown. As little as a 1% dilution of clioquinol in PBS (100%=0.8 μM) or 8 nM clioquinol is capable of doubling the recovery of Aβ in the soluble phase.

In sequential extraction experiments, as described above, clioquinol (1.12 μM) has been shown to result in a 2.5 fold increase in solubilization of Aβ relative to PBS alone (see FIGS. 3A and 3B). Significantly, the findings of the present invention show that very low (8 nM) concentrations of clioquinol may resolubilize more than twice the amount of Aβ compared to PBS buffer alone (see FIGS. 3A and 3B). This suggests that such low concentrations are reasonably expected to be therapeutically effective in treating amyloidosis, preferably that occurring in AD-affected human subjects.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

All patents and publications cited in the present specification are incorporated by reference herein in their entirety.

What is claimed is:

1. A method for the therapy of amyloidosis comprising administering to a patient in need thereof an effective amount of clioquinol.

2. The method of claim 1, wherein the amyloidosis therapy is therapy for Alzheimer's Disease.

3. The method of claim 2, further comprising administering to said patient Vitamin B12 supplement.

4. The method of claim 2, wherein clioquinol is administered intermittently.

5. The method of claim 2, wherein the clioquinol is administered orally.

6. The method of claim 3, wherein the Vitamin B12 is administered orally.

7. The method of claim 3, wherein Vitamin B12 is administered intramuscularly.

8. The method of claim 2, further comprising administering trace metals with or subsequent to the administration of the clioquinol.

9. The method of claim 2, wherein clioquinol is administered parenteraly.

10. The method of claim 2, wherein clioquinol is administered intradermaly.

11. The method of claim 2, wherein the therapy is carried out up to 10 years.
